(19) United States
(12) Patent Application Publication
(10) Pub. No.: US 2013/0027664 A1
(43) Pub. Date: Jan. 31, 2013

(54) OPHTHALMOLOGIC APPARATUS, OPHTHALMOLOGIC PHOTOGRAPHING METHOD, AND PROGRAM

(71) Applicant: Nobuyoshi Kishida, Nishitokyo-shi (JP)

(72) Inventor: Nobuyoshi Kishida, Nishitokyo-shi (JP)

(73) Assignee: CANON KABUSHIKI KAISHA, Tokyo (JP)

(21) Appl. No.: 13/558,247

(22) Filed: Jul. 25, 2012

(30) Foreign Application Priority Data
Jul. 29, 2011 (JP) 2011-167049

(51) Int. Cl. A61B 3/14

(52) U.S. Cl. 351/206; 351/246

(57) ABSTRACT

An ophthalmologic apparatus controls a second light source according to a fundus image corrected using sensitivity as to a first wavelength band and a light quantity of a first light source when a fundus image is captured.

### BACKGROUND OF THE INVENTION

1. Field of the Invention

The present invention relates to an ophthalmologic technology for controlling a light quantity of photography and, more particularly, to a technique for controlling a light quantity from a captured image.

2. Description of the Related Art

Conventionally, there is known an ophthalmologic apparatus that observes an image of a target eye to be examined obtained by irradiating the target eye with light and receiving a reflected light from the target eye, and subsequently obtains an image of the target eye as a still image with a photographic light. Reflectance of lights from target eyes is different for each target eye due to individual differences. Consequently, a technique for appropriately controlling a light quantity of a light which an image sensor receives is needed.

As such a technique, Japanese Patent Application Laid-Open No. 04-150831 discusses a technique for irradiating an target eye with an observation light, and metering the observation light reflected from the target eye, thereby determining a light quantity of a photographic light based on a photometric value.

Furthermore, in recent years, an ophthalmologic apparatus that allows the replacement of an imaging unit having an image sensor according to photographing purpose has been developed. Generally, sensitivity as to a visible photographic light for obtaining a photographic image is adjusted for each imaging unit in conformity with standardized specifications. However, sensitivity characteristics of image sensors for wavelength bands (e.g., R, G, and B) which are different from the photographic light used for observation, are not uniform for each imaging unit.

Further, Japanese Patent Application Laid-Open No. 2006-158822 discusses a technique for detecting a type of replaced digital camera, and setting a segmentation range of an image based on detected information and information stored in a storage unit.

However, when the identical target eye is photographed using various replaceable imaging units, images with different exposures may be obtained. In particular, there is a problem that difference in exposures is likely to appear in non-mydriatic photography that performs observation and photography with different wavelengths.

Further, the technique discussed in Japanese Patent Application Laid-Open No. 2006-158822 allows the acquisition of parameters for each digital camera, but does not allow the acquisition of sensitivity information to perform sensitivity adjustment.

### SUMMARY OF THE INVENTION

The present invention is directed to providing a device for obtaining a light quantity suitable for fundus photography of a target eye to be examined.

According to an aspect of the present invention, an ophthalmologic apparatus includes an illumination optical system configured to have a first light source which emits light with a first wavelength band and a second light source which emits light with a wavelength band different from the first wavelength band, and to illuminate an target eye with light from either one of light sources, an imaging unit configured to capture as a fundus image via an imaging optical system, reflected light from the fundus of the target eye illuminated by the illumination optical system, and a control unit configured to control the second light source according to the fundus image corrected using sensitivity as to the first wavelength band and a light quantity of the first light source when the fundus image is captured.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 is a configuration view of a fundus camera according to an exemplary embodiment.

FIG. 2 illustrates a portion of an operation unit of the fundus camera according to the exemplary embodiment.

FIG. 3 is a flowchart illustrating a flow of processing of the fundus camera according to the exemplary embodiment.

FIG. 4 is a flowchart illustrating a flow of processing for changing a light quantity correction value according to the exemplary embodiment.

FIG. 5 is a flowchart illustrating a flow of processing for photometric value correction according to the exemplary embodiment.

### DESCRIPTION OF THE EMBODIMENTS

Various exemplary embodiments, features, and aspects of the invention will be described in detail below with reference to the drawings.

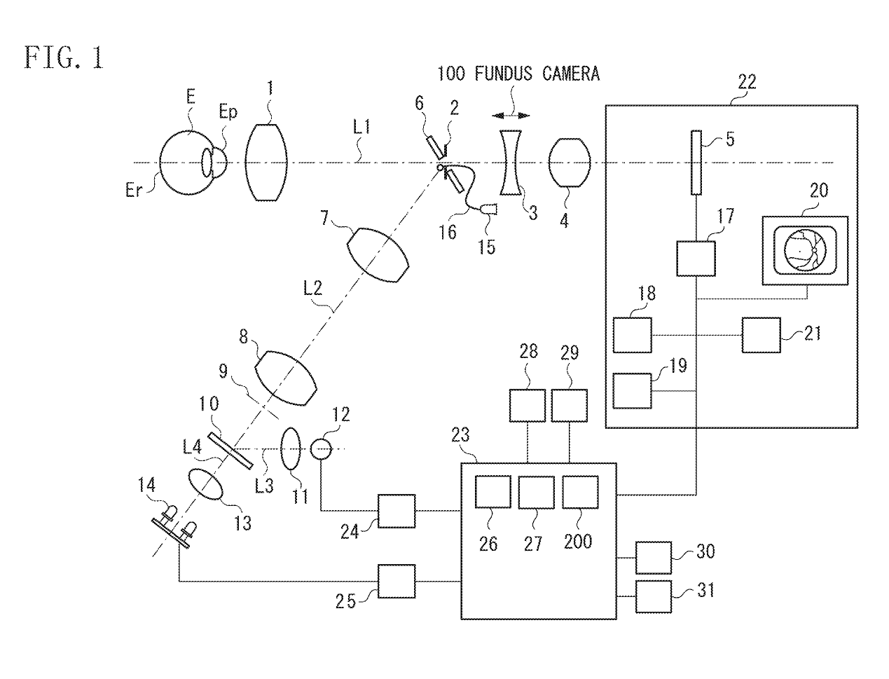

An example configuration of a fundus camera 100 of an ophthalmologic photographing apparatus according to a first exemplary embodiment of the present invention will be described with reference to FIG. 1.

An imaging optical system includes an objective lens 1 arranged facing a target eye E to be examined, a photographic diaphragm 2 provided on an optical axis L1 of the objective lens 1, a focusing lens 3, and an imaging (tube) lens 4, and guides a reflected light at an eye fundus to an image sensor 5. The imaging optical system, when an observation light is radiated, guides a reflected light of the observation light from the eye fundus to the image sensor 5, and when a photographic light is radiated, the imaging optical system guides a reflected light of the photographic light from the eye fundus to the image sensor 5.

An imaging unit 22 includes the image sensor 5 that has sensitivity as to the photographic light and the observation light, an analog-to-digital (A/D) conversion element 17 that A/D converts an output signal of the image sensor 5 to obtain image data, and a memory 18 that stores the image data. The imaging unit 22 further includes a light metering unit 19, a monitor 20, an imaging control unit 21, and is detachably fixed by a mount unit (not illustrated) to a housing of a fundus camera optical unit. An image of the fundus of the target eye is captured by the imaging optical system and the imaging unit 22.

An illumination optical system includes an objective lens 1, a perforated mirror 6, a lens 7, a lens 8, a ring diaphragm 9, a dichroic mirror 10, and a condenser lens 11, a condenser lens 13, and guides the observation light and the photographic light to the target eye E.

The perforated mirror 6 is obliquely provided near the photographic diaphragm 2, and the lens 7 and the lens 8 are arranged on an optical axis L2 in a reflection direction of the perforated mirror 6. Further, to the perforated mirror 6, a Western Digital (WD) light source 15 for projecting alignment indexes onto a cornea Ep of the target eye is connected via a fiber 16. The WD light source 15 is a light source with substantially single wavelength of 700 nm or a very narrow wavelength band. Light sources having other wavelength bands may also be used.

A ring diaphragm 9 having a ring-shaped aperture has a light shielding unit in the center of the optical axis, and is disposed at a position that is optically substantially conjugate with a pupil Ep of the target eye E via the lens 7 and the lens 8. The dichroic mirror 10 has a characteristic that transmits light of a first wavelength band which is a band of the observation light and reflects light of a second wavelength band of the photographic light. The dichroic mirror 10 is arranged on the optical axis L2 together with the ring diaphragm 9. On an optical axis L3 in a reflection direction of the dichroic mirror 10, the condenser lens 11, and a photographing light source 12 as a second light source are arranged. On an optical axis L4 in a transmission direction of the dichroic mirror 10, the condenser lens 13 and an observation light source 14 as a first light source are arranged.

The photographing light source 12 is a light source that irradiates the target eye with, for example, a pulse light for photography. The photographing light source 12 is controlled by a photographing light source control unit 24.

The observation light source 14 has a plurality of light-emitting diodes (LEDs), irradiates the target eye with a stationary light, and is controlled by an observation light source control unit 25. In the present exemplary embodiment, the photographing light source refers to a light source that illuminates the eye fundus in order to capture a target image of fundus. Further, the observation light source refers to a light source that irradiates the target eye in order to perform photographing preparation such as alignment of the fundus camera and the target eye, before capturing an image of target fundus.

Before carrying out the main photographing by irradiating with the photographic light, an examiner observes the eye fundus based on moving pictures of the image of fundus obtained by irradiating with the observation light, and performs positional adjustments or adjustments of focusing and other photographing conditions. The photographing light source 12 in the present exemplary embodiment is a light source with broadband wavelengths of 420 to 750 nm as a second wavelength band. The observation light source 14 is a light source with substantially single wavelength of 850 nm, or 850 nm band having 850 nm in the center as a first wavelength band. By employing an infrared wavelength light source for the observation light source 14, miosis (narrowing) during observation is suppressed.

A fundus illumination optical system, the photographing light source 12 and the observation light source 14 irradiate the target eye with the observation light and the photographic light to illuminate the eye fundus.

The above-described configuration is provided within one housing, and constitutes a fundus camera optical unit. The fundus camera optical unit is mounted on a sliding stand (not illustrated) so that it can be aligned with the target eye E.

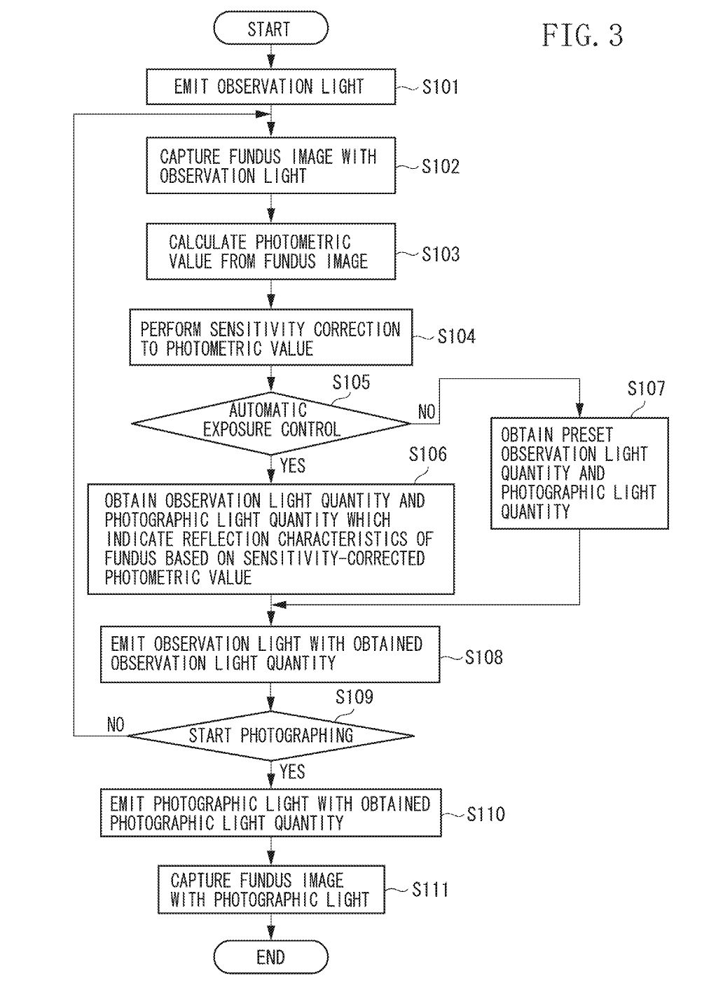

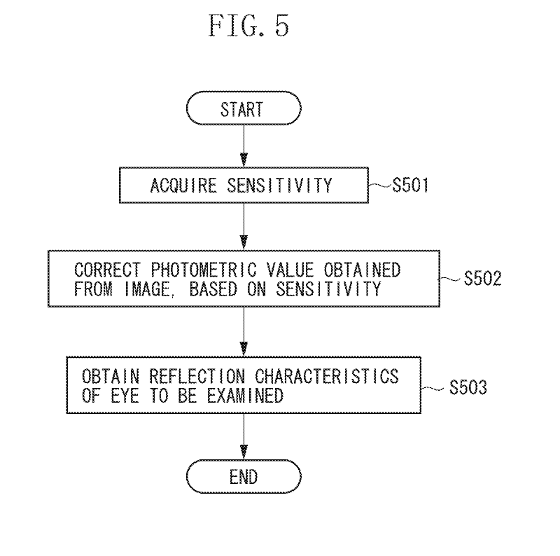

A control unit 23 is provided with a central processing unit (CPU) 101 (not illustrated), and performs an overall control of the fundus camera using a computer program or data stored in a random access memory (RAM) or read only memory (ROM). The processing in the flowcharts in FIG. 3 and FIG. 5 is carried out by execution of the computer program.

Further, the control unit 23 has a photographing light quantity calculation unit 26, an observation light quantity calculation unit 27, and a photometric value correction unit 200. Further, the control unit 23 is connected with the light metering unit 19, an image memory 28, a light quantity memory 29, an operation unit 30, and a photographing switch 31.

The photographing light quantity calculation unit 26, the observation light quantity calculation unit 27, and the photometric value correction unit 200 each determine light quantities of the photographic and observation light sources based on information of the observation light quantity with which the observation light source 14 has irradiated the target eye.

A photographer inputs a photographing light quantity correction value Ff and an observation light quantity correction value Fo at the photographing light quantity correction portion 32 and the observation light quantity correction portion 33. The light quantity herein used refers to the amount of radiation energy radiated within a fixed time length per unit area, and is time integration over a given time Δt of a light flux Φ. In the fundus camera 100 according to the present exemplary embodiment, a shutter (not illustrated) is put in an open state, and the observation light source 14 adjusts a light quantity by adjusting its light intensity.

Further, the control unit 23 adjusts a light quantity of the photographic light which the light metering unit 19 receives, by adjusting the photographing light source 12 such that an integrated value of radiation amounts shows a predetermined value.

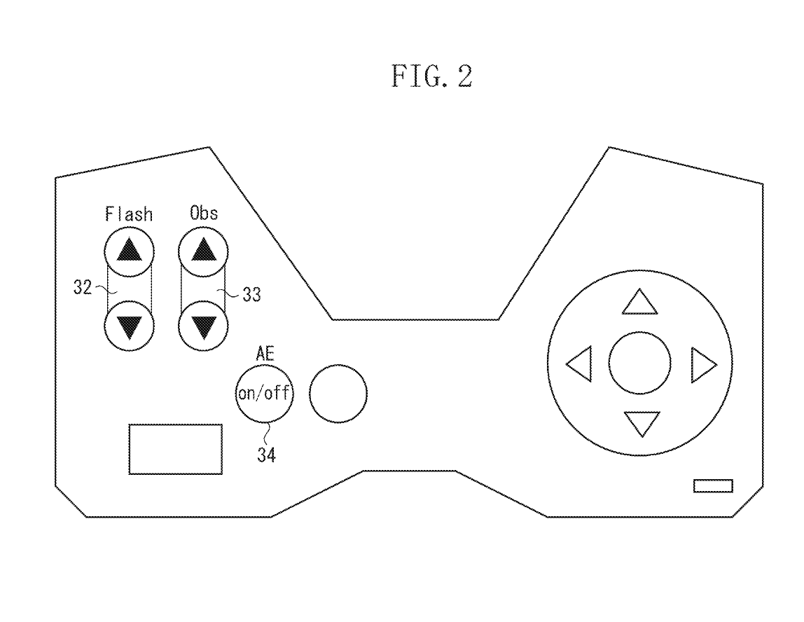

The operation unit 30 is provided to input an instruction from the examiner, and includes at least the photographing light quantity correction portion 32, the observation light quantity correction portion 33, a light quantity control switching portion 34, and a fixation light selection portion 350 (not illustrated). FIG. 2 illustrates a portion of the operation unit 3 in which switches are included. The operation unit 30 is constituted of, for example, a joystick, a dial, and switches. In the present exemplary embodiment, the photographing light quantity correction portion 32, the observation light quantity correction portion 33, and the light quantity control switching portion 34 are switches pressable by the photographer.

The photographing light quantity correction portion 32 is constituted of two independent switches to allow correction to a positive side, and to a negative side of the light quantities, to be used in a light quantity correction in an automatic photographing light quantity control, or light quantity settings in a manual light quantity control. The observation light quantity correction portion 33 is similarly constituted.

The fixation light selection portion 350 selects at least either observation of the optic nerve nipple part, or the entire fundus, and guides a visual direction of the examinee by changing a position at which the fixation light is lighted up in accordance with selected photographing site.

When the optic nerve nipple part is selected, the fixation light is selected and lighted up so that the visual direction is guided such that the optic nerve nipple part comes to the center part of the optical axis of the imaging optical system. When the whole fundus observation is selected, the fixation light is selected and flashed to guide the visual direction so that a region of an intermediate position of the optic nerve nipple part and a macula part comes to the center part of the optical axis of the imaging optical system.

Further, the functions of other operation units in the present exemplary embodiment will be described. First, by tilting a joystick (not illustrated) forward or backward and left or right as viewed from the examiner side, positional relationship in forward or backward and left or right directions between the target eye and the fundus camera can be adjusted. Furthermore, by turning the dial, positional relationship in up or down direction between the target eye and the fundus camera body can be adjusted.

The processing of the fundus camera 100 will be described with reference to the flowchart illustrating the processing described in FIG. 3.

In step S101, the observation light source 14 as the first light source emits an observation light. The observation light emitted by the observation light source 14 illuminates the fundus Er of the target eye E.

A light flux reflected and scattered from the fundus Er illuminated by a light radiated from the observation light source 14 leaves the target eye E from a pupil Ep as a returning light, and reaches the image sensor 5. The imaging unit 22 including the image sensor 5 receives the light which has reached the image sensor 5 and captures an image of the fundus as a fundus image. In this processing, each pixel of the image sensor 5 receives the observation light and outputs an electric signal corresponding to each received light quantity. An output from each pixel is A/D converted by the A/D conversion element 17, and is stored in the memory 18 as fundus image data. Further, the output from the image sensor 5 which has been converted into digital signal, is output to the monitor 20 via the imaging control unit 21. The images stored in the memory 18 may be displayed by the monitor 20. In step S102, fundus observation image is reflected accordingly.

The received light quantity of the observation light source 14 is adjusted by an automatic control by the fundus camera 100, or by the operator operating the operation unit 30, such that the image of fundus reflected onto the monitor 20 yields an appropriate brightness.

The WD light source 15 projects alignment indexes onto a cornea Ep of the examinee's eye via the fiber 16. The operator refers to projected alignment indexes using the joystick (not illustrated), and performs alignment between the target eye E and the fundus camera optical unit.

Furthermore, a position in the optical axis direction of the focusing lens 3 is adjusted by operating a focus knob (not illustrated), and focus adjustment of the projected image of fundus is performed.

In step S103, the light metering unit 19 calculates an average pixel value as a photometric value S, from the images stored in the memory 18.

In this case, a size of the region over which the photometric value S is calculated is varied depending on a site selected by the fixation light selection portion 350. Since reflected light quantity at the optic nerve nipple is larger compared to those at other sites, it may be the case that the photometric value S is calculated from within a region of the optic nerve nipple part. This is because if other regions are included, average value tends to be decreased. In contrast, in the whole fundus observation, it may be the case that a returning light is kept from the whole image during photographing at a constant value. Consequently, the photometric value S is calculated from a range where the eye fundus is captured. The calculated photometric value S is output to the photometric value correction unit 200, the photographing light quantity calculation unit 26, and the observation light quantity calculation unit 27.

A method for defining the observation light quantity and the photographing light quantity, which is a characteristic control according to aspects of the present invention will be described with reference to steps S104 to S107.

In step S104, the photometric value correction unit 200 corrects the photometric value S calculated by the light metering unit 19, in accordance with sensitivity in an observation state of the image sensor 5. The correction method for the photometric value S in step S104 will be described in detail below.

In step S105, the control unit 23 determines automatic light quantity control ON/OFF. As described above, the automatic light quantity control ON/OFF is performed by the light quantity control switching portion 34. If the automatic light quantity control is set to ON (YES in step S105), the process proceeds to step S106, and if the automatic light quantity control is set to OFF (NO in step S105), the process proceeds to step S107.

If the automatic light quantity control is set to ON, in step S106, the photographing light quantity calculation unit 26 as an acquisition unit obtains a ratio of a light quantity of the reflected light from the eye fundus, based on the photometric value S of the observation light obtained by the light metering unit 19 and a value of the light quantity of the observation light of the observation light source 14. Then, the photographing light quantity calculation unit 26 determines the ratio of the light quantity of the reflected light from a fundus, as a fundus reflectance R as the reflection characteristics of the fundus of the target eye.

The photographing light quantity calculation unit 26 stores table information in which fundus reflectance Rs, and photographing light quantities which allow photographing of the eye fundus with proper exposure are associated with each other. The photographing light quantity calculation unit 26 refers to this information and obtains a standard photographing light quantity Pfs which allows photographing of the fundus with proper exposure.

Furthermore, the standard photographing light quantity Pfs is increased or decreased by a photographing light quantity correction value Ff, and the photographing light quantity calculation unit 26 calculates a control photographing light quantity Pf.

In step S106, the photographing light quantity calculation unit 26 refers to the table information in which fundus reflectance Rs, and observation light quantities which allow observation of the fundus with proper exposure are associated with each other, and obtains a standard observation light quantity Pos which allows the observation of the fundus with proper exposure.

Furthermore, the standard observation light quantity Pos is increased or decreased by an observation light quantity correction value Fo, and the photographing light quantity calculation unit 27 calculates a control photographing light quantity Po. The photographing light quantity calculation unit 26, and the observation light quantity calculation unit 27 in the present exemplary embodiment calculate the control photographing light quantity Pf, and the control observation light quantity Po, on the basis of the table information in which reflection characteristics of the fundus, and light quantities which allow observation, and photographing to be performed with proper exposure are associated with each other. In this regard, in a case where regions for obtaining the photometric values S are changed for each site, the tables are prepared for each site. In other words, the tables are prepared for the regions of at least optic nerve nipple part and for the whole image.

In this way, the control photographing light quantity Pf which is a light quantity when photograph is to be taken, is calculated by addition of the standard photographing light quantity Pfs which is automatically defined from reflection characteristics of the fundus of the target eye, and the photographing light quantity correction value Ff.

Further, the control observation light quantity Po which is a light quantity when observation is to be performed, is calculated by addition of the standard observation light quantity Pos which is automatically defined from reflection characteristics of the fundus of the target eye, and the observation light quantity correction value Fo.

If the automatic light quantity control is set to OFF, in step S107, the central control unit 22 substitutes the photographing light quantity correction value Ff into the control photographing light quantity Pf. Further, the central control unit 22 substitutes the observation light quantity correction value Fo into the control photographing light quantity Po.

The process proceeds to step S108 via step S106, or step S107. In step S108, the central control unit 22 stores the control photographing light quantity Pf calculated by the photographing light quantity calculation unit 26, in the light quantity memory 29. Further, the observation light quantity light source control unit 25 controls the observation light source 14 to obtain the control observation light quantity Po calculated by the observation light quantity calculation unit 27, and the observation light source 14 irradiates the target eye E with the observation light according to the control.

Next, in step S109, when alignment and focus adjustment is completed, the examiner presses the photographing switch 31.

In step S110, the photographing light source control unit 24 controls the photographing light source 12 to show the determined light quantity, and the photographing light source 12 irradiates the target eye E with a visible light according to the control. A light flux produced from the photographing light source 12 illuminates the fundus Er of the target eye E.

In step S111, the imaging unit 22 captures an image of the fundus irradiated with the visible light. A light flux is a returning light reflected and scattered from the fundus Er which is illuminated by the light flux produced from photographing light source 12. The light flux leaves the target eye E from the pupil Ep, and reaches the image sensor 5. The image sensor 5 receives this visible light to generate an electronic signal. The A/D conversion element 17 converts the generated signal into digital signal, and the control unit 23 stores this signal in the image memory 28 as still image data.

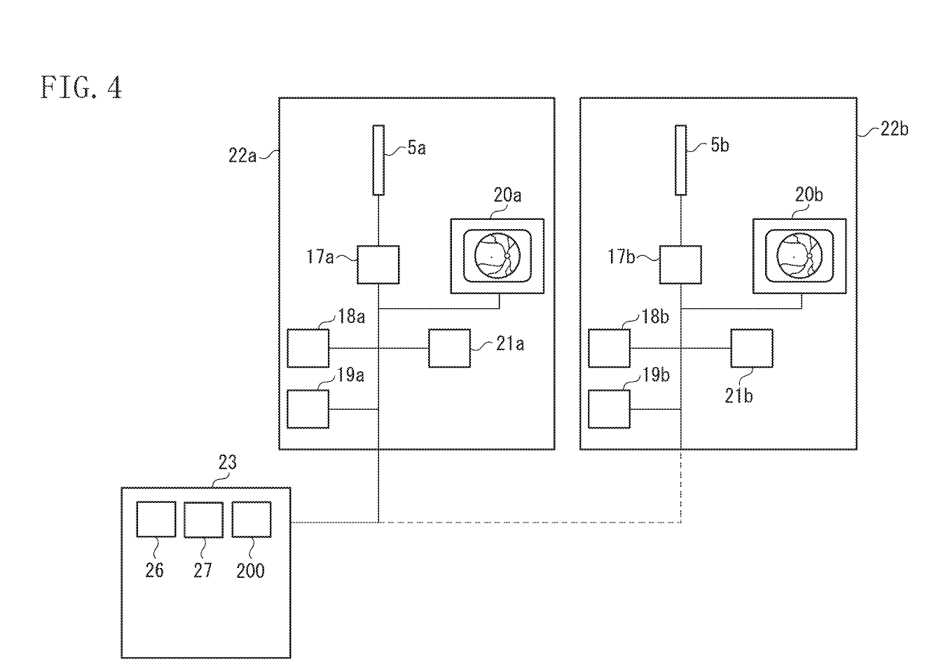

A correction method for the photometric value of the photometric value correction unit 200, which corresponds to step S104 will be described with reference to FIG. 4 and FIG. 5.

FIG. 4 illustrates a configuration in which a plurality of imaging units with different sensitivity characteristics is replaceably attached. An imaging unit 22a and an imaging unit 22b can be attached and detached by a housing of the fundus camera optical unit and a mount unit (not illustrated), and are examples of the imaging unit 22 with different sensitivity characteristics.

The similar components as those in the imaging unit 22 described in FIG. 1 are denoted by the same reference numerals.

As described in steps S101 to S103 in FIG. 3, a photometric value Sa calculated by the light metering unit 19a is calculated based on an observation image captured by the image sensor 5a. Similarly, a photometric value Sb is calculated based on an observation image captured by the image sensor 5b.

At this time, it is assumed, for example, that for the same target eye, the photometric value Sa and the photometric value Sb of the imaging unit 22a and the imaging unit 22b take the same value. As described above in step S106, the control photographing light quantity Pf and the control observation light quantity Po become an identical value. Therefore, the photographing light quantity and the observation light quantity become identical for the same target eye.

Next, the imaging unit 22a and the imaging unit 22b will be described. The imaging unit 22a and the imaging unit 22b are both precisely adjusted to have the same sensitivity as to wavelength bands used in photography of still images. This prevents exposures of photographic images from differing as to the identical sensitivity. Further, since wavelength of light used in observation and photographing are the same, in a case of general use of the imaging unit 22a and the imaging unit 22b, there is no need for adjustment of sensitivity as to wavelength band used in the observation.

However, in a case of the present exemplary embodiment where the wavelength band of the photographing light source 12 and the observation light source 14 are different, sensitivity of the wavelength band used for the observation is not precisely adjusted, and as a result, defects will appear.

This comes from the fact that the photometric value Sa and the photometric value Sb could become different for the same target eye. More specifically, since the photometric value Sa and the photometric value Sb are calculated based on each observation image captured by the image sensor 5a and the image sensor 5b, difference occurs between the photometric value Sa and the photometric value Sb due to different sensitivity in observation.

In particular, in a case of the present exemplary embodiment, a light source with wavelength band of 420 to 750 nm is used as the photographing light source 12, and a light source with wavelength band of 850 nm is used as the observation light source 14. As a result, sensitivity difference will markedly arise.

Consequently, in the ophthalmologic photographing apparatus that performs light quantity control using the photometric value Sa and the photometric value Sb in the observation, images with different exposures will be formed for the same target eye, between the imaging unit 22a and the imaging unit 22b.

In the imaging unit 22a and the imaging unit 22b, the image sensor 5a and the image sensor 5b each are precisely adjusted for sensitivity as to wavelength band with which a still image is captured. On the other hand, difference in sensitivities as to wavelength bands used in observations is not uniform.

For example, if sensitivity as to wavelength band used for observation becomes higher with respect to light with which a still image is captured, the photometric value S becomes larger. Conversely, if the sensitivity is lower, the photometric value S becomes smaller. Therefore, the photometric values for the same target eye will differ, between the imaging unit 22a and the imaging unit 22b. As a result, in addition to photographic images with different exposures, the observation light quantity will differ for similar reason, and accordingly observation images will look differently.

According to aspects of the present invention, the photometric value correction unit 200 is provided in order to solve the defects described above. Next, the processing inside the photometric value correction unit 200 will be described with reference to FIG. 5.

In step S501, the photometric value correction unit 200 acquires a correction value for photometric value as sensitivity information of the imaging unit 22. In the present exemplary embodiment, the photometric correction value can be stored in a nonvolatile RAM or the like serving as a storage unit to back up data. For example, when the imaging unit 22a is used, a value, 1.1 is recorded as the photometric correction value. On the other hand, when the imaging unit 22b is used, a value, 0.9 is recorded as the photometric correction value.

In addition, a method for storing in advance the photometric correction values of the imaging unit 22a and the imaging unit 22b may be considered as a table for photometric correction value. In a case where photographing is performed by frequently replacing the imaging unit 22a and the imaging unit 22b, the photometric correction values of the imaging unit 22a and the imaging unit 22b are stored as the table for photometric correction value, and the imaging unit is automatically identified on the apparatus side, based on identification information of the imaging unit 22a and the imaging unit 22b. In the present exemplary embodiment, the central control unit 22 automatically discriminates between the imaging unit 22a and the imaging unit 22b based on IDs stored inside the imaging units as the identification information.

Then, a calculation method of photometric correction value will be described. In the present exemplary embodiment, in a state where the imaging unit 22a is mounted on the mount portion of the fundus camera optical unit, the reference observation light quantity is radiated from the objective lens 1, and the photometric value Sa is acquired at that time. Next, the photometric value Sa is divided by the reference photometric value So which is a reference of the apparatus, and the photometric correction value as information indicating sensitivity is backed up. In this regard, the reference photometric value S0 is a light quantity which is captured by the imaging unit, when the reference observation light quantity is radiated.

A reflection portion 110 (not illustrated) for inserting and detaching a reflecting member is provided. The reflecting member reflects an illumination light on a common light path of the illumination optical system and the imaging optical system. In a state where the reflecting member is inserted into the common light path, a pixel value of a fundus image captured by the light radiated from the observation light source 14, so that sensitivity from the light quantity of the observation light source 14 are acquired at that time. The common light path used herein means a light path from the objective lens 1 to the diaphragm 2.

For example, while the reflecting member is capped to the objective lens 1, the observation light source 14 irradiates with the reference observation light quantity, and the photometric value Sa is acquired at that time. Further, while the imaging unit 22b is mounted, a photometric correction value as information indicating sensitivity can be calculated, in a similar method.

According to the present exemplary embodiment, in a case where the imaging unit 22 is simplified, it may be the case that that the photometric value correction unit 200 is provided within the housing of the optical unit of the fundus camera 100. On the other hand, it is also conceivable to provide the photometric value correction unit 200 inside the imaging unit 22. The detailed descriptions will be omitted, but in this case, since the photometric value can be used after carrying out correction, the housing of the optical unit of the fundus camera 100 can be simplified.

In either case, the configuration is similar, and arrangement of the photometric value correction unit 200 can be changed depending on the apparatus use.

In step S502, the photometric value S obtained from the light metering unit 19 is multiplied by the photometric correction value as information indicating sensitivity obtained in step S501. The photometric value S as used herein is an average value of fundus images obtained by the imaging unit.

For example, if the photometric value Sa (average value of the fundus images) obtained from the imaging unit 22a is 90, and the photometric value Sb obtained from the imaging unit 22b is 110, the photometric values after the processing in step S502 become 90×1.1=99 and 110×0.9=99, respectively.

In step S503, the photometric value correction unit 200 obtains reflection characteristics of the fundus of the target eye from the photometric value which has been subjected to sensitivity correction in step S502 and the light quantity at the time of photographing. In the present exemplary embodiment, the photometric value S is calculated from an image, but output of the image sensor 5 may be directly corrected using sensitivity information.

As described above, the photometric value correction unit 200 is configured to correct the photometric values using sensitivity. Consequently, even when the photometric value Sa obtained from the imaging unit 22a becomes 90, and the photometric value Sb obtained from the imaging unit 22b becomes 110 for the same target eye, the photometric value S is set to 99 for the processing described in step S105 and beyond in FIG. 3.

Further, the photometric value correction unit 200 is described as a method for correcting the photometric value, so that sensitivity differences between during photographing and during observation are held constant. In other words, when sensitivity as to the wavelength band of the photographic light which has been precisely adjusted is used as the base (reference), it can be presumed that sensitivity differences in observation and photographing are corrected.

In the conventional apparatus in which the photometric value correction unit 200 is not provided, as illustrated in the above-described example, when the photometric value Sa obtained from the imaging unit 22a is 90, and the photometric value Sb obtained from the imaging unit 22b is 110, difference of about 20% which is a ratio of the both photometric values arises, between the photographing light quantity and the observation light quantity. Therefore, for an operator, the apparatus becomes clumsy to use with different brightness in observation images. In addition, for a person reading an image who has received photographic images, the apparatus will become less precise showing different exposures in photographic images.

Through the above-described processing, according to aspects of the present invention, fundus reflection characteristics of the target eye is obtained by performing corrections of a photometric value of the reflected light from the fundus using a photometric value which indicates sensitivity. Thus, a photographing light quantity and an observation light quantity can be calculated which enables photographing and observation with proper exposures even when the imaging unit is changed.

Furthermore, the imaging unit can be automatically identified based on identification information of the imaging unit. Accordingly, without the need to perform special operation, when the operator replaces the imaging unit, observation, and photographing light quantities are automatically controlled to be appropriate light quantities which conform to respective purposes of photographing and observation, and good fundus examination can be performed.

Further, the imaging unit is provided in which a photometric value of the reflected light from the fundus is corrected by using a photometric value indicating sensitivity. Accordingly, without performing special operation, when the operator replaces the imaging unit, observation and photographing light quantities are automatically controlled to be appropriate light quantity which conforms to respective purposes of photographing and observation, and good fundus examination can be performed.

In the present exemplary embodiment, both the reflected light of the observation light and the reflected light of the photographic light are guided to the image sensor 5. A sensor for the first wavelength band, and a sensor for the second wavelength band are similar devices. If the both sensors are similar, an optical member can be shared between them, which makes the imaging unit compact. Further, since the observation system and the photographing system are similar to each other to the point of the sensor portion, a precision is improved when reflection characteristics of the fundus during the observation is used as reflection characteristics for the photographing.

### Other Exemplary Embodiments

In the first exemplary embodiment, the photographing light source 12 is a source with broadband wavelengths of 420 to 750 nm, and the observation light source 14 is a source with substantially single wavelength of 850 nm or very narrow wavelength band, but light sources having other wavelength bands may be used. Further, in a case where wavelength bands of the photographing light source 12 and the observation light source 14 are widely overlapping with each other, it is also acceptable that the both light sources are arranged on the identical optical axis.

In the first exemplary embodiment, a shutter (not illustrated) remains open from pre-irradiation until post-irradiation of the observation light source 14 and the photographing light source 12. The observation light source 14 adjusts its light intensity, and the photographing light source 12 adjusts time length during which the photographic light is emitted. As a result, a light quantity of the photographic light which the sensor receives is adjusted. However, in addition to this method, to obtain a predetermined exposure, a light quantity which the sensor receives may be adjusted by inserting or removing a barrier or a filter on the light path. Further, adjustment of the light quantity which reaches the sensor of the photographing light source 12 may be performed by adjustment of light intensity, adjustment of length of time the shutter is open (shutter speed).

In the first exemplary embodiment, the photographing light quantity correction portion 32 is constituted of two independent switches to deal with inputs of positive correction, and negative correction. However, one switch may perform both positive correction and negative correction, in the form of a slide switch or a rocker switch or the like. Same applies to the observation light quantity correction portion 32.

In the first exemplary embodiment, the functions which the operation unit 30 performs, may be changed by the control of the control unit 23. Alternatively, separate dials, buttons or the like may be provided for each function. Positional adjustment of the fundus camera body which is performed by the joystick, or the dial, may be automatically performed by the control unit 23.

Further, initial values of the photographing light quantity correction value Ff, and the observation light quantity correction value Fo may be set by the operator via a setting unit.

The unit for setting individual/interlocking changes of the photographing, and observation correction values may be provided in the operation unit 30 as a switch or the like.

In the first exemplary embodiment, the fundus camera 100 includes the control unit 23, the image memory 28, the photographing light quantity calculation unit 26, the observation light quantity calculation unit 27, the photometric value correction unit 200, the operation unit 30, the photographing light quantity correction portion 32, the observation light quantity correction portion 33, the light quantity control switching portion 34. However, a part or the whole of the functions realized by the above-described configuration, may be realized by external computers connected with the fundus camera 100.

As described above, even when there is sensitivity difference between the image sensor in the observation which receives the reflected light from the target eye, and the imaging unit in the photographing, the light quantities can be corrected. Consequently, replacement of the digital camera does not have influence on the photographing. As a consequence, photographing with appropriate photographing light quantity is possible and brightness of observation images and photographic images are kept appropriate. Accordingly, there is no need to care about influence of replacement of the digital camera on exposures of the photographic images.

In this way, a mechanism for obtaining a light quantity suitable for the fundus photography of the target eye can be provided.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2011-167049 filed Jul. 29, 2011, which is hereby incorporated by reference herein in its entirety.

What is claimed is:

1. An ophthalmologic apparatus comprising:
an illumination optical system configured to have a first light source which emits light with a first wavelength band and a second light source which emits light with a wavelength band different from the first wavelength band, and to illuminate a target eye to be examined with the light from either one of light sources;
an imaging unit configured to capture as a fundus image via an imaging optical system, reflected light from a fundus of the target eye illuminated by the illumination optical system; and
a control unit configured to control the second light source according to a fundus image corrected using sensitivity as to the first wavelength band and a light quantity of the first light source when the fundus image is captured.

2. The ophthalmologic apparatus according to claim 1, further comprising:
an acquisition unit configured to acquire the sensitivity as to the light with the first wavelength band.

3. The ophthalmologic apparatus according to claim 1, further comprising:
a reflection unit configured to insert or remove a reflecting member which reflects an illumination light, on a common light path of the illumination optical system and the imaging optical system,
wherein the acquisition unit acquires the sensitivity according to a pixel value of the fundus image captured by the light radiated from the first light source while the reflecting member is inserted into the light path, and the light quantity of the first light source at that time.

4. The ophthalmologic apparatus according to claim 1, further comprising:
a storage unit configured to store a sensitivity as to the first wavelength band,
wherein the acquisition unit acquire the sensitivity stored in the storage unit.

5. The ophthalmologic apparatus according to claim 1, further comprising:
a calculation unit configured to obtain reflection characteristics of the fundus of the target eye from a value obtained by correcting a photometric value based on the pixel value of the fundus image using information indicating the sensitivity, and a light quantity of the first light source when the fundus image is captured,
wherein the control unit controls the second light source such that a predetermined light quantity is reflected from the fundus of the target eye, according to the reflection characteristics.

6. The ophthalmologic apparatus according to claim 5, wherein the calculation unit changes a size of a fundus image region from which the photometric value is obtained, according to a photographing site.

7. The ophthalmologic apparatus according to claim 5, wherein the acquisition unit acquires average pixel values of images obtained by the imaging unit, as a photometric value.

8. The ophthalmologic apparatus according to claim 1, wherein the control unit controls light quantities of the first light source based on a table in which reflection characteristics of the fundus of the target eye obtained based on the fundus images corrected using sensitivity as to the first wavelength band, and light quantities of the first light source are associated with each other, when the fundus images are captured.

9. The ophthalmologic apparatus according to claim 1, wherein the control unit controls light quantities of the second light source based on a table in which reflection characteristics of the fundus of the target eye obtained based on fundus images corrected using sensitivity as to the first wavelength band, and light quantities of the second light source are associated with each other, when the fundus images are captured.

10. The ophthalmologic apparatus according to claim 1, wherein the imaging unit captures a fundus as a moving image using the first light source as an observation light, and captures a fundus as a still image using the second light source as a photographic light.

11. The ophthalmologic apparatus according to claim 1, wherein the second wavelength band includes at least a visible wavelength band, and the first wavelength band is infrared.

12. The ophthalmologic apparatus according to claim 1, wherein the second wavelength band is 420 to 750 nm, and the first wavelength band is 850 nm band.

13. The ophthalmologic apparatus according to claim 1, wherein the imaging unit has a sensor for the first wavelength band, and a sensor for the second wavelength band which are similar to each other

14. The ophthalmologic apparatus according to claim 1, wherein the imaging unit is detachably attached to the ophthalmologic apparatus, and can be replaced with other imaging units showing different sensitivities.

15. The ophthalmologic apparatus according to claim 1, wherein the control unit performs control such that an integrated value of flash amounts of the second light source assumes a predetermined value.

16. A photographing method of a fundus comprising:
obtaining a fundus image as an image of an target eye captured by a sensor with an observation light;
acquiring a light quantity of the observation light when the fundus image is captured and sensitivity of the sensor as to the observation light;
obtaining photographing light quantity based on a pixel value of the fundus image, a light quantity of the observation light, and sensitivity of the sensor as to the observation light; and.
imaging the fundus of the target eye with the photographic light having the photographing light quantity.

17. A storage medium storing a program for causing a computer to execute the photographing method according to claim 16.
